(19) United States
(12) Patent Application Publication
(10) Pub. No.: US 2024/0148539 A1
(43) Pub. Date: May 9, 2024

(54) FLUID COLLECTION DEVICES, SYSTEMS, AND METHODS

(71) Applicant: PUREWICK CORPORATION, Covington, GA (US)

(72) Inventors: Nick Austermann, Atlanta, GA (US); Jason Iain Glithero, McDonough, GA (US); Ashley Marie Johannes, Atlanta, GA (US)

(21) Appl. No.: 18/415,080

(22) Filed: Jan. 17, 2024

Related U.S. Application Data
(63) Continuation of application No. 17/051,554, filed on Oct. 29, 2020, now Pat. No. 11,938,053.
(60) Provisional application No. 62/665,321, filed on May 1, 2018.

(51) Int. Cl. A61F 5/451; A61F 5/44

(52) U.S. Cl. CPC A61F 5/451; A61F 5/4408

(57) ABSTRACT

Examples relate to systems, devices, and methods for attaching a fluid collection device to a user or removing fluid collected from a user in the e fluid collection device using a vacuum source operably coupled thereto. The fluid collection devices include urine collection devices shaped to complement the female anatomy near the urethra, attach to the user with one or more flanges, and the vacuum source is operably coupled to the fluid collection device via one or more sections of conduit.

### CROSS-REFERENCE TO RELATED APPLICATIONS

This application is a continuation application of U.S. patent application Ser. No. 17/051,554 filed on 29 Oct. 2020, which is a U.S. Nationalization of PCT International Application No. PCT/US2019/029610 filed on 29 Apr. 2019, which claims priority to U.S. Provisional Application No. 62/665,321 filed on 1 May 2018, the disclosure of each of which is incorporated herein in its entirety by this reference.

### BACKGROUND

An individual may have limited or impaired mobility such that typical urination processes are challenging or impossible. For example, the individual may have surgery or a disability that impairs mobility. In another example, the individual may have restricted travel conditions such as those experience by pilots, drivers, and workers in hazardous areas. Additionally, fluid collection from the individual may be needed for monitoring purposes or clinical testing.

Bed pans and urinary catheters, such as a Foley catheter, can be used to address some of these circumstances. However, bed pans and urinary catheters have several problems associated therewith. For example, bed pans can be prone to discomfort, spills, and other hygiene issues. Urinary catheters be can be uncomfortable, painful, and can cause urinary tract infections.

Thus, users and manufacturers of fluid collection devices continue to seek new and improved devices, systems, and methods to collect urine.

### SUMMARY

Embodiments disclosed herein are related to devices, systems, and methods of using fluid collection devices. In an embodiment, a fluid collection device is disclosed. The fluid collection device includes a fluid collection member. The fluid collection member includes a fluid impermeable barrier at least partially defining a chamber, the fluid impermeable barrier also defining an opening extending therethrough, the opening configured to be positioned adjacent to a female urethra or have a male urethra positioned therethrough. The fluid collection member includes wicking material disposed at least partially within the chamber. The fluid collection member includes a conduit disposed within the chamber, the conduit including an inlet positioned within the fluid collection device and an outlet configured to be in fluid communication with a portable vacuum source. The fluid collection device includes at least one flange extending outwardly from the fluid collection member, the at least one flange including an adhesive member thereon.

In an embodiment, a fluid collection system is disclosed. The fluid collection system includes a fluid storage container configured to hold a fluid. The fluid collection system includes a fluid collection device in fluid communication with the fluid storage container. The fluid collection device includes a fluid collection member. The fluid collection member includes a fluid impermeable barrier at least partially defining a chamber, the fluid impermeable barrier also defining an opening extending therethrough, the opening configured to be positioned adjacent to a female urethra or have a male urethra positioned therethrough. The fluid collection member includes a wicking material disposed at least partially within the chamber. The fluid collection member includes a conduit disposed within the chamber, the conduit including an inlet positioned within the fluid collection device and an outlet configured to be in fluid communication with a portable vacuum source. The fluid collection device includes at least one flange extending outwardly from the fluid collection member, the at least one flange including an adhesive member thereon. The fluid collection system includes a vacuum source in fluid communication with one or more of the fluid storage container or the fluid collection device, the vacuum source configured to draw fluid from the fluid collection device.

In an embodiment, a method to collect fluid is disclosed. The method includes positioning an opening of a fluid collection device adjacent to a female urethra or around a male urethra of a user, the opening defined by a fluid impermeable barrier of the fluid collection device. The method includes positioning securing the fluid collection device to the user. The method includes positioning receiving fluid from the female urethra or male urethra into a chamber of the fluid collection device, the chamber of the fluid collection device at least partially defined by the fluid impermeable barrier.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1 is a block diagram of a system for fluid collection, according to an embodiment.

FIG. 2A is an isometric view of a fluid collection device, according to an embodiment.

FIG. 2B is a front view of a user with the fluid collection device of FIG. 2A positioned for use, according to an embodiment.

FIGS. 3-5 are schematic cross-sectional views of fluid collection devices taken along the plane A-A of FIG. 2A, according to embodiments.

FIGS. 6-8 are schematic cross-sectional views of fluid collection devices taken along the plane B-B of FIG. 2A, according to embodiments.

FIG. 9A is an isometric view of a fluid collection device, according to an embodiment.

FIG. 9B is schematic cross-sectional view of the fluid collection device taken along the plane C-C of FIG. 9A, according to an embodiment.

FIG. 10 is a flow diagram of a method to collect fluid, according to an embodiment.

### DETAILED DESCRIPTION

Embodiments disclosed herein are related to devices, systems, and methods of using fluid collection devices and systems. The devices, systems, and methods of using fluid collection devices and systems include at least one flange extending from the fluid collection device where the at least one flange is positioned to attach to the skin of a user and align the fluid collection device with one or more anatomical structures of the user (e.g., urethra). The devices, systems, and methods of using fluid collection devices and systems include a portable vacuum source to remove urine from the fluid collection device. The portable vacuum source may allow for portable usage of the systems and methods herein such as in non-hospital environments.

In an embodiment, a fluid collection device includes a fluid impermeable barrier that at least partially defines a chamber. The fluid impermeable barrier also defines an opening extending therethrough that is configured to be positioned adjacent to a female urethra or have a male urethra positioned therethrough. The fluid collection device also includes a tube having a channel extending between an inlet and outlet thereof. The inlet is configured to be coupled to a suction source and the outlet is configured to be fluidly coupled to (e.g., in fluid communication with) a fluid storage (vessel or container). The outlet is positioned downstream from the inlet. The channel also defines at least one aperture therein that fluidly couples an interior of the channel to the rest of the chamber.

The fluid collection devices disclosed herein are configured to collect fluid(s) from an individual. The fluid(s) collected by the fluid collection devices can include urine. The fluid(s) collected by the fluid collection devices can also include at least one of vagina discharge, penile discharge, reproductive fluids, blood, sweat, or other bodily fluids.

The fluid collection devices disclosed herein are configured to be used in fluid collection systems. The fluid collection systems disclosed herein include a gas source. Systems that include a gas source can, in some embodiments, resolve several problems associated with systems that include a vacuum source. For example, a system that includes a vacuum source draws fluid(s) towards the vacuum source and deposits most of the fluid(s) in a fluid storage container before the fluid(s) can reach the vacuum source. However, a small quantity of fluid(s) (e.g., vapor from the fluid) can still reach the vacuum source, which can contaminate and/or damage (e.g., rust) the vacuum source. Additionally, a large quantity of the fluid(s) can reach the vacuum source when the fluid storage container is substantially full. However, a system that includes a gas source moves the fluid(s) away from the gas source, thereby preventing contamination and/or damage. For example, a gas source may be used to create a vacuum by flowing a gas past a connected end of the conduit at a perpendicular or oblique angle to the conduit to create a vacuum in the conduit. The fluids are pulled up the conduit and into the gas flow in the direction of the gas flow, which is away from the gas source. In another embodiment, systems that include a vacuum source cannot be used in environments that do not include an available vacuum source (e.g., the environment does not include a vacuum source or the vacuum source is being used). As such, systems that include a gas source can be used in environments that do not include an available vacuum source. A liquid source can be used to create and implement a vacuum in the same way as the gas source. The vacuum source or gas source can be utilized with any of the devices or systems disclosed herein to remove a fluid therefrom.

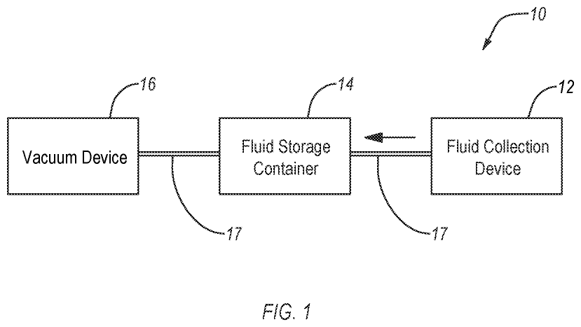

FIG. 1 is a block diagram of a system 10 for fluid collection, according to an embodiment. The system 10 includes a fluid collection device 12, a fluid storage container 14, and a vacuum source 16. The fluid collection device 12, the fluid storage container 14, and the vacuum source 16 may be in fluid communication with (e.g., fluidly coupled to) each other via one or more conduits 17. For example, fluid collection device 12 may be in fluid communication with one or more of the fluid storage container 14 or the vacuum source via the conduit 17. Fluid (e.g., urine or other bodily fluids) collected in the fluid collection device 12 may be removed from the fluid collection device 12 via the conduit 17 which protrudes into an interior region of the fluid collection device 12. For example, a first open end of the conduit 17 may extend into the fluid collection device 12 to a reservoir therein. The second open end of the conduit 17 may extend into the fluid storage container 14 or the vacuum source 16. The suction force may be introduced into the interior region of the fluid collection device 12 via the first open end of the conduit 17 responsive to a suction (e.g., vacuum) force applied at the second end of the conduit 17. The suction force may be applied to the second open end of the conduit 17 by the vacuum source 16 either directly or indirectly.

The suction force may be applied indirectly via the fluid storage container 14. For example, the second open end of the conduit 17 may be disposed within the fluid storage container 14 and an additional conduit 17 may extend from the fluid storage container 14 to the vacuum source 16. Accordingly, the vacuum source 16 may apply suction to the fluid collection device 12 via the fluid storage container 14. The suction force may be applied directly via the fluid storage container 14. For example, the second open end of the conduit 17 may be disposed within the vacuum source 16. An additional conduit 17 may extend from the vacuum source 16 to a point outside of the fluid collection device 12, such as to the fluid storage container 14. In such examples, the vacuum source 16 may be disposed between the fluid collection device 12 and the fluid storage container 14.

The fluid collection device 12 may be shaped and sized to be positioned adjacent to a female urethra. For example and as described in more detail below, the fluid collection device 12 may include a fluid collection member and at least one flange positioned and equipped to attach to the skin of a user and align the fluid collection member in a selected portion of the anatomy of the user (e.g., adjacent to or on the urethra or vagina). For example, the at least one flange may include an adhesive for reversibly attaching to the skin of the user and may be positioned on the fluid collection member to align an opening of the fluid collection member with the vagina (e.g., on or over the urethra or between the labia) of a female user.

The fluid collection member of the fluid collection device 12 may include a fluid impermeable barrier at least partially defining a chamber (e.g., interior region of the fluid collection device member) of the fluid collection device 12. The fluid impermeable barrier also defines an opening extending therethrough from the external environment. The opening may be positioned on the fluid collection member to be aligned adjacent to a female urethra. The fluid collection member of the fluid collection device 12 may include a fluid permeable membrane disposed within the fluid impermeable barrier. The fluid collection member of the fluid collection device 12 may include a fluid permeable support disposed within the fluid permeable membrane. The conduit 17 may extend into the fluid collection device 12 at a first end region, through one or more of the fluid impermeable barrier, fluid permeable membrane, or the fluid permeable support to a second end region of the fluid collection member of the fluid collection device 12. Exemplary fluid collection devices for use with the systems and methods herein are described in more detail below.

In examples, the fluid storage container 14 may include a bag (e.g., drainage bag), a bottle or cup (e.g., collection jar), or any other enclosed container for storing bodily fluid(s) such as urine. In examples, the conduit 17 may extend from the fluid collection device 12 and attach to the fluid storage container 14 at a first point therein. An additional conduit 17 may attach to the fluid storage container 14 at a second point thereon and may extend and attach to the vacuum source 16. For example, the fluid storage container 14 may include a container in fluid communication with a first conduit section that is also in fluid communication with the fluid collection member of the fluid collection device 12. The container may be in fluid communication with a second section of the conduit 17 that is also in fluid communication with a vacuum source. In such examples, the vacuum source 16 may provide a vacuum/suction through the container to the fluid collection member to provide suction in the chamber of the fluid collection member. Accordingly, a vacuum (e.g., suction) may be drawn through fluid collection device 12 via the fluid storage container 14. As the fluid is drained from the chamber, the fluid may travel through the first section of conduit to the fluid storage container where it may be retained. Fluid, such as urine, may be drained from the fluid collection device 12 using the vacuum source 16.

In some examples, the vacuum source 16 may include a portable vacuum source. In examples, the portable vacuum source may be disposed in or on the fluid collection device 12. In such examples, the conduit 17 may extend from the fluid collection device and attach to the (portable) vacuum source 16 at a first point therein. An additional conduit 17 may attach to the vacuum source 16 at a second point thereon and may extend out of the fluid collection device 12, and may attach to the fluid storage container 14. Accordingly, a vacuum (e.g., suction) may be drawn through fluid collection device 12 via the fluid storage container 14.

The vacuum source 16 may include one or more of a vacuum line plumbed into patient care facility, a manual vacuum pump, and electric vacuum pump, a diaphragm pump, a centrifugal pump, a displacement pump, a magnetically driven pump, a peristaltic pump, or any pump configured to produce a vacuum. The vacuum source 16 may provide a vacuum or suction to remove fluid from the fluid collection member of the fluid collection device 12. In examples, the vacuum source 16 may be powered by one or more of a power cord (e.g., connected to a power socket), one or more batteries, or even manual power (e.g., a hand operated vacuum pump). In examples, the vacuum source 16 (e.g., portable vacuum source) may be sized and shaped to fit outside of, on, or within the fluid collection device 12. For example, the vacuum source 16 (e.g., portable vacuum source) may include one or more miniaturized pumps or one or more micro pumps. The vacuum sources 16 disclosed herein may include one or more of a switch, a button, a plug, a remote, or any other device suitable to activate the vacuum source 16. It should be understood that the vacuum sources 16 disclosed herein may provide a portable means of providing a suction or vacuum that allows use of the devices and systems herein outside of hospital or care facility environments where vacuum lines are plumbed into patient rooms or large (e.g., larger or heavier than a patient can readily carry) vacuum sources are located. For example, a portable vacuum source may be small and light enough to be carried by a user (e.g., patient) or aid (e.g., nurse) during transportation of the user.

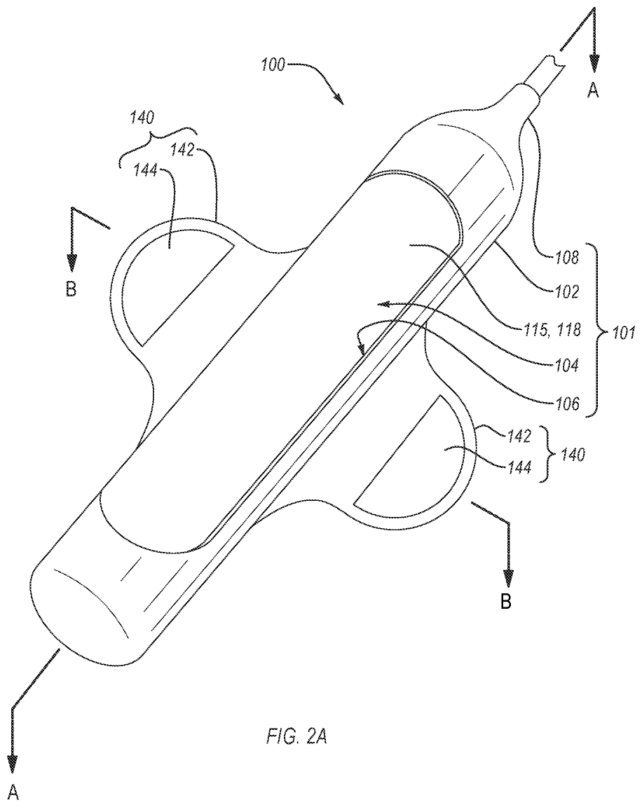

FIG. 2A is an isometric view of a fluid collection device 100, according to an embodiment. The fluid collection device 100 includes a fluid collection member 101 and at least one flange 140 extending from the fluid collection member 101. In some examples, the fluid collection member 101 may be substantially cylindrical, ellipsoid, prismatic, or any other shape suitable for complementing or contouring to the vaginal region of a female subject. The fluid collection member 101 may include a fluid impermeable barrier 102, wicking material 115, and a conduit 108. The wicking material 115 may be disposed at least partially within the fluid impermeable barrier 102. The conduit 108 may be at least partially disposed with wicking material 115.

The fluid impermeable barrier 102 at least partially defines at least a portion of an outer surface of the fluid collection member 101. The fluid impermeable barrier 102 at least partially defines a chamber 104 therein (e.g., interior region of the fluid collection member 101) and an opening 106. The opening 106 is formed in and extends through the fluid impermeable barrier 102, thereby enabling fluid(s) to enter the chamber 104 from outside of the fluid collection member 101 of the fluid collection device 100. The opening 106 can be configured to be positioned adjacent to a female urethra, such as between the labia majora of a female user.

The fluid impermeable barrier 102 may also temporarily retain or store fluid(s) in the chamber 104. For example, the fluid impermeable barrier 102 can be formed of any suitable fluid impermeable materials, such as a fluid impermeable polymer (e.g., silicone, polypropylene, polyethylene, polyethylene terephthalate, polyurethane, a polycarbonate, polyvinyl chloride, latex, silicone, etc.), a metal film, another suitable material, or combinations thereof. As such, the fluid impermeable barrier 102 may prevent at least some of the fluid(s) from exiting the portions of the chamber 104 that are spaced from the opening 106.

In an embodiment, the fluid impermeable bather 102 can be air permeable and fluid impermeable. In such an embodiment, the fluid impermeable barrier 102 can be formed of a hydrophobic material that defines a plurality of pores. In an example, one or more portions of at least an outer surface of the fluid impermeable barrier 102 can be formed from a soft and/or smooth material thereby reducing chafing of the skin of the user. The fluid impermeable barrier 102 may include markings thereon, such as one or more markings to aid a user in aligning the device 100 on the wearer. For example, a line on the fluid impermeable barrier 102 (e.g., opposite the opening 106) may allow a healthcare professional to align the opening 106 over the urethra of the wearer. In examples, the markings may include one or more of alignment guide or an orientation indicator, such as a stripe or hashes. Such markings may be positioned to align the device 100 to one or more anatomical features such as a pubic bone, etc.

The wicking material 115 may be disposed at least partially within the fluid impermeable barrier 102. The wicking material 115 may include permeable material designed to wick or allow fluid to pass therethrough. The permeable properties referred to herein can be wicking, capillary action, diffusion, or other similar properties or processes, and are referred to herein as “permeable” and/or “wicking.” Such “wicking” may not include absorption into the wicking material. The wicking material 115 may collect the fluid that travels through the opening 106. The wicking material 115 may include more than one material, such as a plurality of materials. The plurality of materials may include a plurality of layers concentrically disposed within one another. The concentrically disposed layers of wicking materials may exhibit a gradient of wicking, such as where the innermost wicking material includes the greatest or least wicking ability of the plurality of materials.

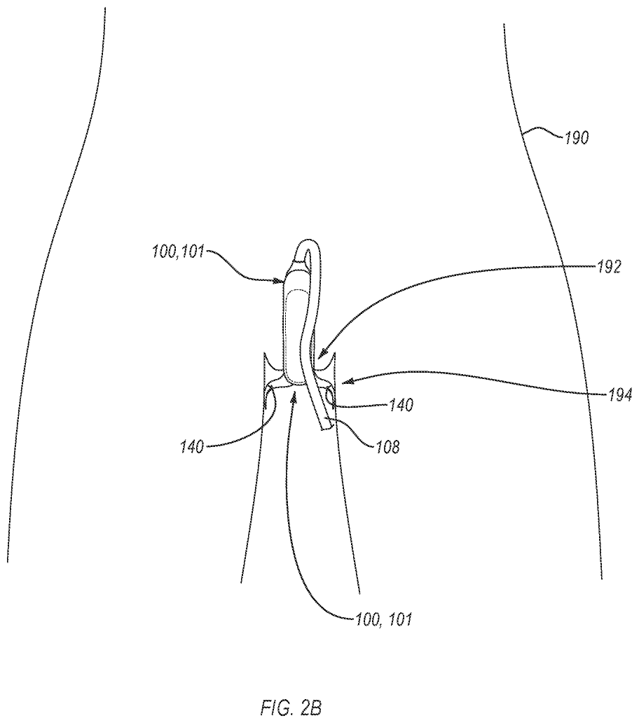

In examples, the wicking material 115 may include one or more of a fluid permeable support (FIGS. 3-8) or a fluid permeable membrane 118. For example, the fluid collection member 101 of the fluid collection device 100 can include a fluid permeable membrane 118 disposed in the chamber 104. The fluid permeable membrane 118 can cover at least a portion (e.g., all) of the opening 106. The fluid permeable membrane 118 can be configured to wick any fluid away from the opening 106, thereby preventing the fluid from escaping the chamber 104. The fluid permeable membrane 118 can also wick the fluid generally towards an interior of the chamber 104, as discussed in more detail below. The fluid permeable membrane 118 can include any material that can wick the fluid. For example, the fluid permeable membrane 118 can include fabric, such as a gauze (e.g., a silk, linen, polyester, or cotton gauze), another soft fabric (e.g., jersey knit fabric or the like), or another smooth fabric (e.g., rayon, satin, or the like). In some examples, the fluid permeable membrane 118 can include an open cell foam. Forming the fluid permeable membrane 118 from gauze, soft fabric, and/or smooth fabric can reduce chafing caused by the fluid collection device 100.

The fluid collection device 100 can include a fluid permeable support 120 (FIGS. 3-8) disposed in the chamber 104. The fluid permeable support 120 may support the fluid permeable membrane 118 since the fluid permeable membrane 118 can be formed from a foldable, flimsy, or otherwise easily deformable material. For example, the fluid permeable support 120 can be positioned such that the fluid permeable membrane 118 is disposed between the fluid permeable support and the fluid impermeable barrier 102. As such, the fluid permeable support 120 can support and maintain the position of the fluid permeable membrane 118. The fluid permeable support 120 can be formed from any fluid permeable material that is less deformable than the fluid permeable membrane 118. For example, the fluid permeable support 120 can include a porous nylon structure or an open cell foam. In embodiments, the fluid permeable support can be omitted from the fluid collection device 100.

In some embodiments, the wicking material 115 (e.g., one or more of the fluid permeable membrane 118 or the fluid permeable support) can at least substantially completely fill portions of the chamber 104 that are not occupied by the conduit 108. For example, the wicking material 115 may fill the portions of the chamber 104 that are not occupied by the conduit 108. In some examples, the fluid permeable membrane 118 and the fluid permeable support may not substantially completely fill the portions of the chamber 104 that are not occupied by the conduit 108. In such examples, the fluid collection device 100 may include the reservoir (e.g., void space) disposed in the chamber 104. The reservoir may include a void space between the wicking material in the chamber 104 and the interior surface of the fluid impermeable barrier 102. At least some of the fluid absorbed by the wicking material 115 may drain out of the wicking material 115 and collect in the reservoir.

The fluid collection member 101 of the fluid collection device 100 may also include conduit 108 that is at least partially disposed in the chamber 104. The conduit 108 may include a flexible material such as plastic tubing (e.g., medical tubing). Such plastic tubing may include a thermoplastic elastomer, polyvinyl chloride, ethylene vinyl acetate, polytetrafluoroethylene, etc., tubing. In examples, the conduit 108 may include silicon or latex. The conduit 108 (e.g., a tube) includes an inlet at a first end region and an outlet at a second end region positioned downstream from the inlet. The conduit 108 places an interior region of the chamber 104 in fluid communication with one or more of the fluid storage container (FIG. 1) or the vacuum source (FIG. 1). The fluid may be removed from the chamber 104 via the conduit 108. As suction or vacuum force is applied or formed in the conduit 108 by the vacuum source (FIG. 1), the fluid in the chamber 104 may be drawn into the inlet and out of the fluid collection member 101 via the conduit 108.

In examples, the conduit 108 may be disposed in an innermost or gravimetrically low spot in the chamber 104. For example, the conduit 108 may extend far enough into the chamber 104 to position the inlet in a gravimetrically low spot of the chamber 104 (e.g., fluid reservoir within the interior of the fluid collection member 101).

The fluid collection member 101 and components thereof may be deformable (e.g., bendable) responsive to pressure applied thereto. For example, the fluid collection member 101 and the components thereof may bend to conform to the surface of the user, such as when disposed between a garment and the user. In examples, the fluid collection member 101 may bend when disposing proximate to the urethra (e.g., between the labia) when undergarments are pulled on over the fluid collection member 101.

The at least one flange 140 may extend from the fluid collection member 101. The at least one flange may include a flange body 142 and an adhesive member 144 disposed on the flange body 142. In some examples, the at least one flange 140 may include 2 or more flanges (e.g., 4 flanges). The at least one flange 140 may include a first flange body extending a first direction away from the fluid collection member 101 and a second flange body extending away from the fluid collection member 101 in a second direction, wherein the first and second directions are substantially opposite one another (e.g., at least 120° apart). The at least one flange 140 may extend along at least a portion of the longitudinal length of fluid collection member 101. For example, the at least one flange 140 may have a width, as viewed parallel to the plane B-B, of least 5% (e.g., at least 10%, at least 20%, at least 30%, at least 40%, at least 50%, or less than 60%) of the longitudinal length of the fluid collection member 101. In examples, the width of the flange 140 may be at least 1 cm, such as 2 cm, 5 cm, 10 cm, 15 cm, 20 cm, 30 cm, or in a range between any combination of the foregoing. The flange 140 (e.g., flange body 142) may extend at least 1 cm away from the fluid collection member 101, such as 1 cm, 3 cm, 5 cm, 10 cm, 15 cm, 20 cm, 25 cm, 30 cm, or in a range between any combination of the foregoing, away from the fluid collection member 101.

In examples, the flange body 142 of the at least one flange 140 may be formed of the same material as the fluid impermeable barrier 102. In examples, the flange body 142 of the at least one flange 140 may be formed from one or more of cloth, paper, plastic, or any other material suitable for deforming responsive to pressure applied thereto and able to withstand moisture without breaking down. For example, the flange body 142 may be formed from thermoplastic elastomer, polyethylene, polyvinyl chloride, ethylene vinyl acetate, polytetrafluoroethylene, latex, silicon, fabric, woven cloth, etc. In examples, the conduit 108 may include silicon or latex. The flange body 142 of the at least one flange 140 may be flexible and may conform to manipulation or movement by a user. The flange body 142 may exhibit any of the dimensions disclosed above for the flange 140.

The flange body 142 may have the adhesive member 144 disposed thereon, such as at a distal portion thereof (e.g., distal from the fluid collection member 101). The adhesive member 144 may be affixed to the flange body 142 by mechanical means such as a staple(s), a clip, hook and loop fasteners, etc.; may be affixed by an adhesive; or may be integrally formed in the flange body 142. The adhesive member 144 may be positioned on the flange body 142 in a position effective to allow the adhesive to be applied to a garment of the skin of the user to maintain a position of the fluid collection member 101 with respect to one or more anatomical features (e.g., proximate to the female urethra or between the labia) of the user. For example, the adhesive member 144 may be positioned on the flange body 142 to allow the at least one flange(s) 140 to adhere to the inner thigh of a user and maintain a position of the associated fluid collection member 101 between the labia of the user 190. In examples, the adhesive member 144 may be flexible or otherwise configured to conform to the anatomical features of the user and accommodate movement of the user.

FIG. 2B is a front view of a user 190 with the fluid collection device 100 positioned for use, according to an embodiment. The user 190 may be a female and the fluid collection device 100 may be positioned for use (e.g., collecting urine or other bodily fluids from the vagina). For example, the flanges 140 may be disposed on the fluid collection member 101 in a position to locate the fluid collection member 101 between the labia 192 when the at least one flange 140 is adhered to the inner thigh 194 of the user 190. In examples, the at least one flange 140 can be adhered to the pubic region (e.g., lower abdominal region) of the user 190 to align the fluid collection member over the urethra of the user 190. In some examples, the at least one flange 140 can be adhered to the fabric of a garment worn by the user 190 (e.g., an undergarment). The conduit 108 may be in fluid communication with the fluid storage container or vacuum pump (not shown) to remove any collected fluid(s) from the fluid collection device 100.

Returning to FIG. 2A, the at least one flange body 142 may include the adhesive member 144 disposed thereon. For example, the adhesive member 144 may be facing upward on the flange body 142 (e.g., in the general direction of the fluid collection member 101). In some examples, the flange 140 may additional or alternatively include the adhesive member 144 facing downward on the flange body 142. In examples, the adhesive member 144 may be located on a distal portion of the flange body 142 (e.g., distal from the fluid collection member 101). In examples, the adhesive member 144 may cover at least 5% of the surface area of the flange body 142, such as 5%, 10%, 20%, 30%, 40%, 50%, 60%, 70%, 80%, 90%, or ranges between any combination of the foregoing, of the surface area of the flange body 142.

In examples, the adhesive member 144 may include a glue, contact adhesive, epoxy, hydrogel adhesive, tape, or other adhesive suitable for attaching the flange to skin or fabric. For example, the adhesive may include an acrylate (e.g., methacrylate or epoxy diacrylate) or any other adhesive suitable for use on bandages.

The cross-sectional profiles of the fluid collection devices disclosed herein may vary. For example, a longitudinal cross section is taken along the plane A-A and an axial cross section may be viewed along the plane B-B in FIG. 2A.

FIG. 3 is a schematic cross-sectional view of the fluid collection device 100 taken along the plane A-A of FIG. 2A, according to an embodiment. The fluid collection device 100 is an example of a female fluid collection device 100 sized, shaped, and otherwise configured to receive fluid(s) from a female user. The fluid collection device 100 includes the fluid collection member 101 and the at least one flange 140. The fluid collection member 101 includes the fluid impermeable barrier 102. The fluid impermeable barrier 102 at least partially defines the chamber 104 (e.g., interior region) and the opening 106. The opening 106 is formed in and extends through the fluid impermeable barrier 102, thereby enabling fluid(s) to enter the chamber 104 from outside of the fluid collection device 100. The opening 106 can be configured to be positioned adjacent to a female urethra. The opening 106 can be positioned on an upward facing portion of the fluid collection member 101 (e.g., region substantially opposite the flanges 140). The fluid collection device 100 also includes conduit 108 that is at least partially disposed in the chamber 104. The conduit 108 (e.g., a tube) includes an inlet 110 at a first end region and an outlet 112 at a second end region positioned downstream from the inlet 110. The conduit 108 places the chamber 104 in fluid communication with the fluid storage container (not shown) or the vacuum source (not shown).

In the illustrated embodiment, the conduit 108 is at least partially disposed in the chamber 104. For example, the conduit 108 may extend into the fluid impermeable barrier 102 from the first end region (e.g., proximate to the outlet 112) and may extend to the second end region (e.g., opposite the first end region) to a point proximate to the reservoir 122 such that the inlet 110 is in fluid communication with the reservoir 122. The fluid collected in the fluid collection member 101 may be removed from the interior region of the chamber 104 via the conduit 108. The conduit 108 may include a flexible material such as plastic tubing (e.g., medical tubing) as disclosed herein. In some examples, the conduit 108 may include one or more portions that are resilient, such as to by having one or more of a diameter or wall thickness that allows the conduit to be flexible.

The fluid collection member 101 may be positioned proximate to the female urethra (e.g., on or between the labia) and urine may enter the chamber 104 of the fluid collection member 101 via the opening 106. The fluid collection member 101 receives the fluid(s) into the chamber 104 via the opening 106. For example, the opening 106 can exhibit an elongated shape that is sized and positioned to extend from a first location below the urethral opening (e.g., at or near the anus or the vaginal opening) to a second location above the urethral opening (e.g., at or near the clitoris or the mons pubis). The opening 106 can exhibit an elongated shape since the space between the legs of a female is relatively small when the legs of the female of closed thereby only permitting the flow of the fluid(s) along a path that corresponds to the elongated shape of the opening 106. The longitudinal axis or dimension of the fluid collection device refers to the axis or dimension that is parallel to largest dimension of the device, such as axially along a cylindrical device as show in FIG. 2A. The opening 106 in the fluid impermeable barrier 102 can exhibit a width that is measured transverse to the longitudinal direction and may be at least about 10% of the circumference of the fluid collection member 101, such as about 25%, 30%, 40%, 50%, 60%, 75%, 85%, 100% or ranges between any combination of the foregoing, of the circumference of the fluid collection member 101. The opening 106 can exhibit a width that is greater than 50% of the circumference of the fluid collection member 101 since the vacuum (e.g., suction) through the conduit 108 pulls the fluid into the conduit 108. In some embodiments, the opening 106 may be vertically oriented (e.g., having a major axis parallel to the longitudinal axis of the device 100). In some embodiments, (not shown), the opening 106 may be horizontally oriented (e.g., having a major axis perpendicular to the longitudinal axis of the device 100). In an example, one or more portions of the fluid impermeable barrier 102 can be configured to be attached to the individual, such as adhesively attached (e.g., with a hydrogel adhesive) to the individual. According to an embodiment, a suitable adhesive for the impermeable barrier (or flanges) is a hydrogel layer, such as those disclosed in U.S. Patent Application Publication No. 2017/0189225, the disclosure of which is incorporated herein by reference in its entirety.

The fluid collection member 101 includes the wicking material 115 disposed in the chamber 104. The wicking material 115 may include one or more of the fluid permeable membrane 118 and the fluid permeable support 120, each disposed in the chamber 104. The fluid permeable membrane 118 can cover at least a portion (e.g., all) of the opening 106. The fluid permeable membrane 118 can be configured to wick any fluid away from the opening 106 thereby preventing the fluid from escaping the chamber 104. The fluid permeable membrane 118 can also wick the fluid generally towards an interior of the chamber 104, as discussed in more detail below. The fluid permeable membrane 118 can include any material that can wick the fluid. For example, the fluid permeable membrane 118 can include fabric, such as a gauze (e.g., a silk, linen, polymer based materials such as polyester, or cotton gauze), another soft fabric, or another smooth fabric. Forming the fluid permeable membrane 118 from gauze, soft fabric, and/or smooth fabric can reduce chafing caused by the fluid collection member 101.

The fluid permeable support 120 may be disposed in the chamber 104, such as concentrically within the fluid permeable membrane 118. The fluid permeable support 120 may be formed from material that is more rigid (e.g., less deformable) than the fluid permeable membrane 118, such as any of the materials disposed herein for a fluid permeable membrane. For example, the fluid permeable support 120 can include a porous nylon structure. The fluid permeable support 120 is configured to support the fluid permeable membrane 118 since the fluid permeable membrane 118 can be formed from a foldable, flimsy, or otherwise easily deformable material. For example, the fluid permeable support 120 can be positioned such that the fluid permeable membrane 118 is disposed between the fluid permeable support 120 and the fluid impermeable barrier 102. As such, the fluid permeable support 120 can support and maintain the position of the fluid permeable membrane 118. In an embodiment, the fluid permeable support 120 can be omitted from the fluid collection member 101.

In an embodiment, the fluid permeable membrane 118 and the fluid permeable support 120 can at least substantially completely fill the portions of the chamber 104 that are not occupied by the conduit 108. In another example, the fluid permeable membrane 118 and the fluid permeable support 120 may not substantially completely fill the portions of the chamber 104 that are not occupied by the conduit 108. In such an example, the fluid collection device 100 includes the reservoir 122 disposed in the chamber 104. The reservoir 122 is a substantially unoccupied portion of the chamber 104. The fluid(s) that is in the chamber 104 can flow through the fluid permeable membrane 118 and/or fluid permeable support 120 to the reservoir 122. The reservoir 122 can store at least some of the fluid(s) therein.

The fluid impermeable barrier 102 can store fluid(s) in the reservoir 122. The reservoir 122 may be disposed in any portion of the interior region of the chamber 104. For example, the fluid reservoir 122 may be positioned in the second end region of the chamber 104.

In an example, the reservoir 122 can be located at the portion of the chamber 104 that is closest to the inlet 110 (e.g., the second end region). However, the reservoir 122 can be located at different locations in the chamber 104. For example, the reservoir 122 can be located at the end of the chamber 104 that is closest to the outlet 112. In another example, fluid collection device 100 can include multiple reservoirs, such as a first reservoir that is located at the portion of the chamber of the chamber 104 that is closest to the inlet 110 (e.g., second end region) and a second reservoir that is located at the portion of the of the chamber 104 that is closest to the outlet 112 (e.g., first end region). In another example, the fluid permeable support 120 is spaced from at least a portion of the conduit 108 and the reservoir 122 can be the space between the fluid permeable support 120 and the conduit 108.

Other examples of fluid impermeable barriers, fluid permeable membranes, fluid permeable supports, chambers, and their shapes and configurations are disclosed in U.S. patent application Ser. No. 15/611,587 filed on Jun. 1, 2017 and U.S. patent application Ser. No. 15/260,103 filed on Sep. 8, 2016 (published as US 2016-0374848 on Dec. 29, 2016), the disclosure of each of which is incorporated herein, in its entirety, by this reference.

The fluid impermeable barrier 102, the fluid permeable membrane 118 and the fluid permeable support 120 can be configured to have the conduit 108 at least partially disposed in the chamber 104. For example, at least one of the fluid permeable membrane 118 and the fluid permeable support 120 can be configured to form a space that accommodates the conduit 108. In another example, the fluid impermeable barrier 102 can define an aperture 124 sized to receive the conduit 108 (e.g., at least one tube). The at least one conduit 108 can be disposed in the chamber 104 via the aperture 124 in the first end region of the device 100. The aperture 124 can be configured to form an at least substantially fluid tight seal against the conduit 108 or the at least one tube thereby substantially preventing the fluid(s) from escaping the chamber 104. In some embodiments, the aperture 124 may be disposed on the second end region nearer the reservoir 122. In such embodiments, the conduit 108 may be disposed in only the second end region with the inlet 110 being disposed in the second end region (e.g., the reservoir 122).

As previously discussed, the conduit 108 is configured to be coupled to, and at least partially extend between, one or more of the fluid storage container (not shown) and the vacuum source (not shown). In an example, the conduit 108 is configured to be directly connected to the vacuum source (not shown). In such an example, the conduit 108 can extend from the fluid impermeable barrier 102 by at least one foot, at least two feet, at least three feet, or at least six feet. In another example, the conduit 108 is configured to be indirectly connected to at least one of the fluid storage container (not shown) and the vacuum source (not shown). In some embodiments, the conduit is secured to a wearer's skin with a catheter securement device, such as a STATLOCK® catheter securement device available from C. R. Bard, Inc., including but not limited to those disclosed in U.S. Pat. Nos. 6,117,163; 6,123,398; and 8,211,063, the disclosures of which are all incorporated herein by reference in their entirety.

The inlet 110 and the outlet 112 are sized, positioned, or otherwise configured to place (e.g., directly or indirectly) the vacuum source (not shown) in fluid communication with the chamber 104 (e.g., the reservoir 122). In an example, the inlet 110 and/or the outlet 112 can form a male connector. In another example, the inlet 110 and/or the outlet 112 can form a female connector. In an example, the inlet 110 and/or the outlet 112 can include ribs that are configured to facilitate secure couplings. In an example, the inlet 110 and/or the outlet 112 can form a tapered shape. In an example, the inlet 110 and/or the outlet 112 can include a rigid or flexible material.

Locating the inlet 110 at or near a gravimetrically low point of the chamber 104 enables the conduit to receive more of the fluid(s) than if inlet 110 was located elsewhere and reduce the likelihood of pooling (e.g., pooling of the fluid(s) can cause microbe growth and foul odors). For instance, the fluid(s) in the fluid permeable membrane 118 and the fluid permeable support 120 can flow in any direction due to capillary forces. However, the fluid(s) may exhibit a preference to flow in the direction of gravity, especially when at least a portion of the fluid permeable membrane 118 and/or the fluid permeable support 120 is saturated with the fluid(s).

As the vacuum source (FIG. 1) applies a vacuum/suction in the conduit 108, the fluid(s) in the chamber 104 (e.g., at the second end region such as in the reservoir 122) may be drawn into the inlet 110 and out of the fluid collection member 101 of the fluid collection device 100 via the conduit 108.

In an example, the conduit 108 is configured to be at least insertable into the chamber 104. In such an example, the conduit 108 can include one or more markers (not shown) on an exterior thereof that are configure to facilitate insertion of the conduit 108 into the chamber 104. For example, the conduit 108 can include one or more markings thereon that are configured to prevent over or under insertion of the conduit 108, such as when the conduit 108 defines an inlet 110 configured to be disposed in or adjacent to the reservoir 122. In another example, the conduit 108 can include one or more markings thereon that are configured to facilitate correct rotation of the conduit 108 relative to the chamber 104. In an example, the one or more markings can include a line, a dot, a sticker, or any other suitable marking. In some examples, the conduit may be frosted or opaque (e.g., black) to obscure visibility of the fluid(s) therein.

In an example, one or more components of the fluid collection device 100 can include an antimicrobial material, such as an antibacterial material where the fluid collection device may contact the wearer or the bodily fluid of the wearer. The antimicrobial material can include an antimicrobial coating, such as a nitrofurazone or silver coating. The antimicrobial material can inhibit microbial growth, such as microbial growth due to pooling or stagnation of the fluid(s). In an example, one or more components (e.g., impermeable barrier 102, conduit 108, etc.) of the fluid collection device 100 can include an odor blocking or absorbing material such as a cyclodextrine containing material or a thermoplastic elastomer (TPE) polymer.

The at least one flange 140 can be disposed on a lower portion of the fluid collection member 101 (e.g., substantially opposite the opening 106). For example, the at least one flange 140 may extend from the fluid collection member 101 may have the at least one flange 140 affixed to or integrally formed in the fluid impermeable barrier 102.

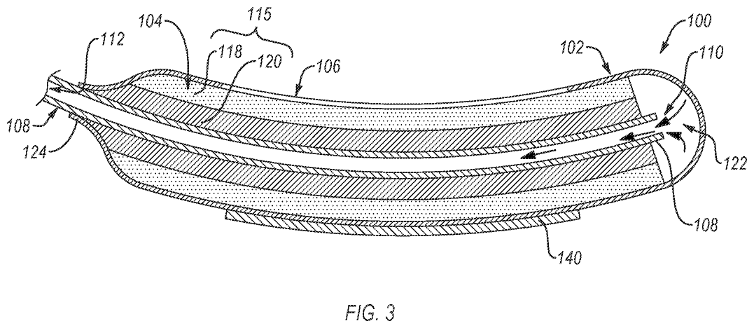

FIG. 4 is a schematic cross-sectional view of the fluid collection device 400 taken along the plane A-A of FIG. 2A, according to an embodiment. The fluid collection device 400 may include the portable vacuum source 330 disposed therein. Except as otherwise disclosed herein, the fluid collection device 400 can be the same as or substantially similar to the fluid collection device 100 of FIG. 2A, in one or more aspects. The fluid collection device 400 can include fluid collection member 401 and the at least one flange 140. The fluid collection member 401 may be similar or identical to the fluid collection member 101 in one or more aspects, such as including one or more of the fluid impermeable barrier 102 that defines the chamber 104 and the opening 106, the wicking material 115, the fluid permeable membrane 118, the fluid permeable support 120, and the reservoir 122, or the conduit 108. The fluid collection device 400 includes the portable vacuum source 330 disposed therein. The portable vacuum source 330 may be similar or identical to the vacuum source 16 as disclosed herein, in one or more aspects. The portable vacuum source 330 may be sized to fit on or within the fluid collection device. The portable vacuum source may be sized and shaped for a person to carry. As shown, the portable vacuum source 330 may be at least partially disposed within the fluid impermeable barrier 102. While a portable vacuum source 330 is depicted in FIG. 4, a fixed vacuum source (e.g., vacuum line) may alternatively or additional be used with the fluid collection device 400.

The fluid collection device 400 includes the conduit 108 that is at least partially disposed in the chamber 104. For example, the wicking material 115 (e.g., the fluid permeable membrane 118, the fluid permeable support 120) may fill a portion of the chamber 104 and leave a portion vacant thereby forming the reservoir 122 between the wicking material 115 and the fluid impermeable barrier 102. The conduit 108 can include one or more walls that define an inlet 110 and the outlet 112. The inlet 110 enables at least some of the fluid(s) that is present in the chamber 104 to enter the conduit 108. In an example, the conduit 108 can be configured to have the inlet 110 located at, near, or spaced at a gravimetrically low point of the chamber 104. In an example, the conduit 108 can be configured to have the at least one inlet 110 disposed in or adjacent to the reservoir 122. As shown the conduit 108 can extend through at least a portion of the chamber 104, such as longitudinally through at least a portion of the wicking material 115 in a concentrically central region fluid collection member 115.

The conduit 108 can be in fluid communication with the interior region (e.g., reservoir 122) of the chamber 104 via the fluid impermeable barrier 102. As such, the fluid impermeable barrier 102 can define the aperture 124. In an example, as illustrated, the aperture 124 enables the conduit 108 to extend outwardly from the chamber 104 when the conduit 108 is only partially disposed in the chamber 104. In examples, the conduit 108 may include a plurality of separate sections. For example and as shown, the conduit 108 may include a first section A and section B. The first section A may include the inlet 110 extending from the distal end (e.g., first end region) to the portable vacuum source 330 and the B section may extend from the portable vacuum source 330 out of the aperture 124, such as to a fluid storage container (not shown).

The portable vacuum source 330 may include any of the portable vacuum pumps disclosed herein. For example, the portable vacuum source 330 may include a manual vacuum pump, a diaphragm pump, a centrifugal pump, a displacement pump, a magnetically driven pump, a peristaltic pump, or any pump configured to produce a vacuum. The portable vacuum source 330 may be sized to fit in the chamber 104 inside of the fluid impermeable barrier 102. In examples, the portable vacuum source 330 may sealed in a fluid tight housing or container. The portable vacuum source 330 may apply a vacuum (e.g., suction) in the A section of the conduit 108 effective to suction fluid from the chamber 104. The fluid may travel through the A section to the B section (e.g., through the portable vacuum source 330) and out of the fluid collection device 300 via the B section by flow induced by the vacuum or suction applied by the portable vacuum source 330. For example, the portable vacuum source 330 may include a centrifugal pump and an impeller therein may draw the fluid from the chamber 104 via the inlet 110 and force the fluid out of the chamber 104 via the B section of the conduit 108. Each of the A section and the B section of the conduit 108 may be in fluid communication with (e.g., sealed) the portable vacuum source 330. In some examples, the portable vacuum source 330 and the conduit 108 can be integrally formed together (e.g., exhibit single piece construction).

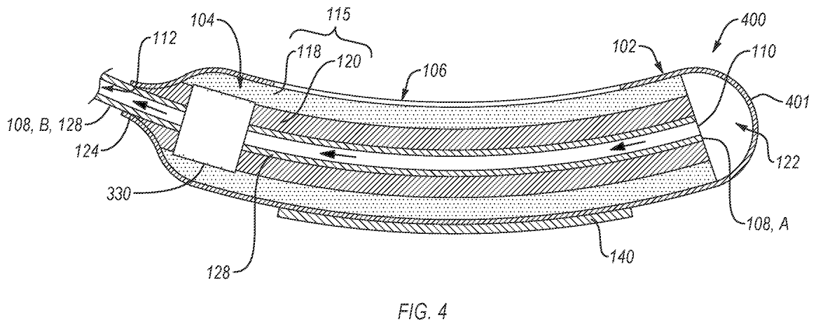

FIG. 5 is a schematic cross-sectional view of the fluid collection device 500 taken along the plane A-A of FIG. 2A, according to an embodiment. The fluid collection device 500 may include the portable vacuum source 330 disposed thereon. Except as otherwise disclosed herein, the fluid collection device 500 can be the same as or substantially similar to the fluid collection device 100 of FIG. 2A, in one or more aspects. The fluid collection device 500 can include fluid collection member 501 and the at least one flange 140. The fluid collection member 501 may be similar or identical to the fluid collection member 101 in one or more aspects, such as including one or more of the fluid impermeable barrier 102 that defines the chamber 104 and the opening 106, the wicking material 115, the fluid permeable membrane 118, the fluid permeable support 120, and the reservoir 122, or the conduit 108. The fluid collection member 501 of the fluid collection device 500 includes the portable vacuum source 330 disposed thereon. The portable vacuum source 330 may be attached to the fluid collection device 500 at or on the fluid impermeable barrier 102. While a portable vacuum source 330 is depicted in FIG. 5, a fixed vacuum source (e.g., vacuum line) may alternatively or additional be used with the fluid collection device 500.

The fluid collection device 500 includes the conduit 108 that is at least partially disposed within the fluid collection member 501. For example, the wicking material 115 (e.g., the fluid permeable membrane 118, the fluid permeable support 120) may fill a portion of the chamber 104 and leave a portion vacant thereby forming the reservoir 122 between the wicking material 115 and the fluid impermeable barrier 102. As shown the conduit 108 can extend through at least a portion of the chamber 104, such as longitudinally through at least a portion of the wicking material 115 in a concentrically central region fluid collection member 115 to the reservoir 122. The conduit 108 may extend through the wicking material 115 to the reservoir 122. The conduit 108 can include one or more walls that define an inlet 110 and the outlet 112. The inlet 110 enables at least some of the fluid(s) that is present in the chamber 104 to enter the conduit 108. In an example, the conduit 108 can be configured to have the inlet 110 located at, near, or spaced at a gravimetrically low point of the chamber 104. In an example, the conduit 108 can be configured to have the at least one inlet 110 disposed in or adjacent to the reservoir 122.

The conduit 108 can be in fluid communication with the interior region of the chamber 104 via the fluid impermeable barrier 102. As such, the fluid impermeable barrier 102 can define the aperture 124. In an example, as illustrated, the aperture 124 enables the conduit 108 to extend outwardly from the chamber 104 when the conduit 108 is only partially disposed in the chamber 104. In examples, the conduit 108 may include a plurality of separate sections. For example and as shown, the conduit 108 may include the first section A and the second section B. The first section A may include the inlet 110 extending from the distal end (e.g., first end region), out of the aperture 124, to the portable vacuum source 330 mounted thereto. The portable vacuum source 330 may be mounted to the outer surface of the fluid collection device 400. The B section may be attached to and extend from the portable vacuum source 330, such as to a fluid storage container (not shown).

The portable vacuum source 330 may include any of the portable vacuum pumps disclosed herein such as a manual vacuum pump, a diaphragm pump, a centrifugal pump, a displacement pump, a magnetically driven pump, a peristaltic pump, or any pump configured to produce a vacuum. The portable vacuum source 330 may be sized to fit in the chamber 104 inside of the fluid impermeable barrier 102. In examples, the portable vacuum source 330 may sealed in a fluid tight housing or container. The portable vacuum source 330 may apply a vacuum (e.g., suction) in the A section of the conduit 108 effective to suction fluid from the chamber 104. The fluid may travel through the A section out of the fluid collection device 400 to the portable vacuum source 330. The fluid may be removed from the portable vacuum source 330 via the B section by flow induced by the vacuum or suction applied by the portable vacuum source 330. For example, the portable vacuum source 330 may include a centrifugal pump and an impeller therein may draw the fluid from the chamber 104 via the inlet 110 and suction the fluid out of the chamber 104 via the portable vacuum source 330 to the B section of the conduit 108. Each of the A section and the B section of the conduit 108 may be in fluid communication with (e.g., sealed) the portable vacuum source 330. In some examples, the portable vacuum source 330 and the conduit 108 (e.g., one or both of the A section or the B section) can be integrally formed together to exhibit single piece construction.

The fluid collection devices shown in FIGS. 2-5 are examples of female fluid collection devices that are configured to collect fluid(s) from females (e.g., collect urine from a female urethra). However, the devices, systems, and methods disclosed herein can include male fluid collection devices shaped, sized, and otherwise configured to collect fluid(s) from males (e.g., a cup shaped fluid collection member to collect urine from a male urethra). In such examples, the flanges of the male fluid collection device may be located on the male fluid collection device to position and maintain the fluid collection device over the male urethra (e.g., penis). In any of the embodiments disclosed herein the conduits 108 may include or be operably coupled to a flow meter (not shown) to measure the flow of fluid(s) therein, one or more securement devices (e.g., a STATLOCK® securement device, not shown) or fittings to secure the conduit 108 to one or more components of the systems or devices disclosed herein (e.g., vacuum source or fluid storage container), or one or more valves to control the flow of fluid(s) in the systems and devices herein.

In an example, at least one of portion of the conduit 108 of the fluid collection devices or systems herein can be formed of an at least partially opaque material which can obscure the fluid(s) that is present therein. For example, the B section of the conduits 108 disclosed herein may be formed of an opaque material or translucent material while the A section may be formed of a transparent material or translucent material. In examples, the B section may include transparent or translucent material. Unlike the opaque or nearly opaque material, the translucent material allows a user of the devices and systems herein to visually identify fluid(s) or issues that are inhibiting the flow of fluid(s) within the conduit 108.

In any of the example, systems or devices disclosed herein, the system of fluid collection device may include moisture sensors (not shown) disposed inside of the chamber of the fluid collection device. In such examples, the moisture sensor may be operably coupled to a controller or directly to the vacuum source, and may provide electrical signals indicating that moisture is or is not detected in one or more portions of the chamber. The moisture sensor(s) may provide an indication that moisture is present, and responsive thereto, the controller of the vacuum source (e.g., vacuum device with a programmable controller) may direct the initiation of suction to the chamber to remove the fluid therefrom. Suitable moisture sensors may include capacitance sensors, volumetric sensors, potential sensors, resistance sensors, frequency domain reflectometry sensors, time domain reflectometry sensors, or any other suitable moisture sensor. In practice, the moisture sensors may detect moisture in the chamber and may provide a signal to the controller of the vacuum source to activate the vacuum source.

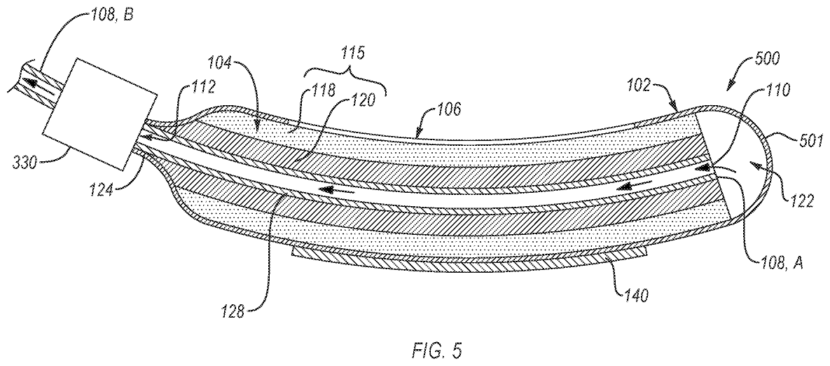

As noted above, the at least one flange 140 (e.g., pair of flanges) may be positioned on the fluid collection member in any of a number of positions and may extend therefrom at any angle. The cross-sectional shape of the fluid collection members disclosed herein may include any of various shapes or sizes. For example, the cross-sectional shape (along the plane B-B of FIG. 2A) may be substantially round (e.g., circular), elliptical, rectangular, triangular, irregular (e.g., having no specific shape), etc. FIGS. 6-8 are schematic cross-sectional views of female fluid collection devices taken along the plane B-B of FIG. 2, according to different embodiments.

FIG. 6 is a schematic cross-sectional view of the fluid collection device 100 taken along the plane B-B of FIG. 2A, according to an embodiment. The fluid collection device 100 includes the fluid collection member 101 and the at least one flange 140 extending therefrom. As shown, the fluid collection member 101 includes the fluid impermeable barrier 102, the wicking member 115 (e.g., the fluid permeable membrane 118 and the fluid permeable support 120), and the conduit 108, all concentrically arranged. As shown, the cross-sectional shape of the fluid collection member 101, and one or more components therein, may be generally round (e.g., circular or ellipsoid).

The at least one flange 140 may extend substantially tangentially from the fluid collection member 101 (e.g., the fluid impermeable barrier 102). For example, the at least one flange 140 may include at least 2 flanges 140, each extending from the fluid collection member 101 tangentially in substantially opposite directions from one another. “Substantially tangentially” may include flanges 140 that extend outwardly at an angle within 10% of 0° from an attachment point of the flange(s) on the outer surface of the fluid impermeable barrier. In examples, the at least one flange 140 may attach to the fluid collection member 101 at a point substantially opposite the opening 106. The fluid collection member 101 may extend toward the user past the flanges 140. Such arrangements may allow the fluid collection member 101 to be positioned on the region of the female urethra, such as on or between the labia.

In examples, the flange bodies 142 may have the adhesive member 144 bound thereto on a surface thereof that generally faces the same direction as the opening 106. In some examples, the flanges 140 may alternatively or additionally include adhesive members 144 on the surface of the flange body 142 that face generally away from the opening 106. The adhesive members 144 may allow the flange body 142 to be attached (e.g., temporarily adhered) to one or more skin surfaces of the subject that are adjacent to the urethra of the user, such as the thighs or pubic region. The adhesive members 144 may secure and maintain the fluid collection device 100 in a position to collect fluid from the user, such as between the labia to collect urine from a female subject.

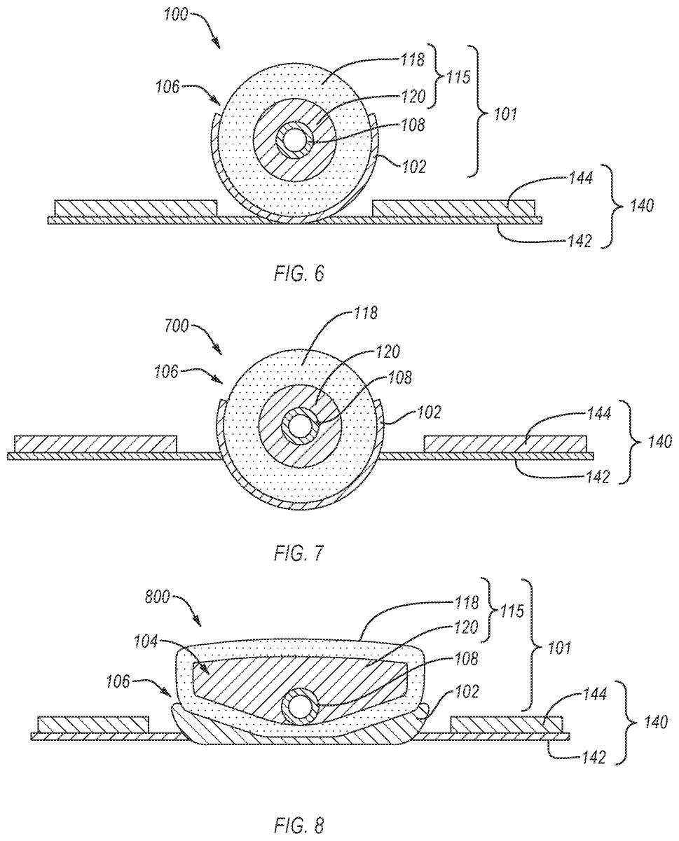

FIG. 7 is a schematic cross-sectional view of the fluid collection device 700 taken from a view along the plane B-B of FIG. 2A, according to an embodiment. The fluid collection device 100 includes the fluid collection member 101 and the at least one flange 140 extending therefrom. As shown, the fluid collection member 101 includes the fluid impermeable barrier 102, the wicking member 115 (e.g., the fluid permeable membrane 118 and the fluid permeable support 120), and the conduit 108, all concentrically arranged. As shown, the cross-sectional shape of the fluid collection member 101, and one or more components therein, may be generally round (e.g., circular or ellipsoid).

The at least one flange 140 may extend substantially perpendicularly away from the fluid collection member 101 (e.g., the fluid impermeable barrier 102). For example, the at least one flange 140 may include at least 2 flanges 140, each extending from the fluid collection member 101 perpendicularly (e.g., in a radial direction) from the fluid impermeable barrier 102 and in substantially opposite directions from one another. “Substantially perpendicularly” may include flanges 140 that extend outwardly at an angle within 10% of 90° with respect to an outer surface of the fluid impermeable barrier at attachment point of the flange(s) 140 thereon. For examples and as shown, the at least one flange 140 may attach to the fluid collection member 101 and radially extend therefrom in radially opposite directions. The fluid collection member 101 may extend toward the user past the flanges 140. Such arrangements may allow the fluid collection member 101 to be positioned on the region of the female urethra, such as on or between the labia.

In examples, the flange bodies 142 may have the adhesive member 144 bound thereto on a surface thereof that generally faces the same direction as the opening 106. In some examples, the flanges 140 may alternatively or additionally include adhesive members 144 on the surface of the flange body 142 that face generally away from the opening 106. The adhesive members 144 may allow the flange body 142 to be attached (e.g., temporarily adhered) to one or more skin surfaces of the subject that are adjacent to the urethra of the user, such as the thighs or pubic region. The adhesive members 144 may secure and maintain the fluid collection device 100 in a position to collect fluid from the user, such as between the labia to collect urine from a female subject.

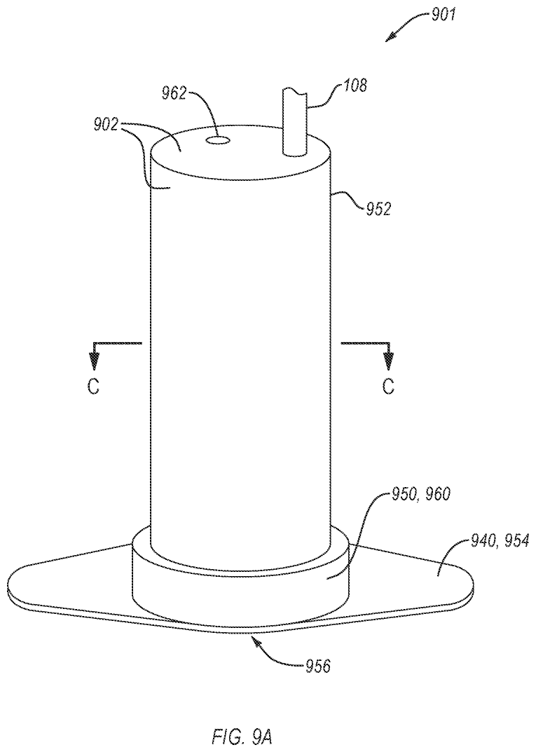

The cross-sectional shape of the fluid collection member and the position(s) of the components therein may vary. FIG. 8 is a schematic cross-sectional view of the fluid collection device 800 taken from a view along the plane B-B of FIG. 2A, according to an embodiment. The fluid collection device 800 may be similar or identical to the fluid collection device 100, in one or more aspects. The fluid collection device 800 includes the fluid collection member 801 and the at least one flange 140 extending therefrom. The fluid collection member 801 may be similar or identical to the fluid collection member 101, in one or more aspects. For example, the fluid collection member 801 includes the fluid impermeable barrier 102, the wicking member 115 (e.g., the fluid permeable membrane 118 and the fluid permeable support 120), and the conduit 108. The cross-sectional shape of the fluid collection member 801 may be generally rectangular (e.g., with rounded corners) as shown, or elliptical. In such examples, the fluid collection member 801 may provide coverage (e.g., fluid absorption) of a larger surface area than the fluid collection member 101 (FIG. 6). In examples, the fluid impermeable barrier 102 may be shaped to provide a relatively wide cross-sectional shape. As shown, the fluid impermeable barrier 102 may form the chamber 104 having a gravimetrically low portion, such as in a V-notched or dished shape. The wicking material 115 may fill at least a portion of the chamber 104. For example, the fluid permeable support 120 may fill an interior portion of the chamber 104, and the fluid permeable member 118 may extend around at least a portion of the fluid permeable support 120. In examples, the fluid permeable support 120 may include region therein for accommodating the conduit 108. In examples, the conduit 108 may be disposed in the gravimetrically low point or region of the chamber 104. As shown, one or more of the conduit 108, fluid permeable member 118, or the fluid permeable support 120 may be non-concentrically disposed (e.g., not centered) in the fluid collection member 801 or fluid impermeable barrier 102.

Additional or alternative shapes for the fluid collection members are considered. For example, the fluid collection member may have a substantially triangular cross-sectional shape where a corner or apex of the triangle faces the user.

The at least one flange 140 may extend substantially perpendicularly away from the fluid collection member 801 (e.g., the fluid impermeable barrier 102). For example, the at least one flange 140 may include at least 2 flanges 140, each extending from the fluid collection member 801 perpendicularly from the fluid impermeable barrier 102 and in substantially opposite directions from one another. For examples and as shown, the at least one flange 140 may attach to the fluid collection member 801 in radially opposite directions. The fluid collection member 801 may extend toward the user past the flanges 140. Such arrangements may allow the fluid collection member 801 to be positioned on the region of the female urethra, such as on or between the labia.

In examples, the flange bodies 142 may have the adhesive member 144 bound thereto on a surface thereof that generally faces the same direction as the opening 106. In some examples, the flanges 140 may alternatively or additionally include adhesive members 144 on the surface of the flange body 142 that face generally away from the opening 106. The adhesive members 144 may allow the flange body 142 to be attached (e.g., temporarily adhered) to one or more skin surfaces of the subject that are adjacent to the urethra of the user, such as the thighs or pubic region. The adhesive members 144 may secure and maintain the fluid collection device 100 in a position to collect fluid from the user, such as between the labia to collect urine from a female subject.

In some examples, the fluid collection devices herein may include more than one conduit therein. the more than one conduit may be disposed in a plurality of regions therein. In examples, conduit 108 may include a plurality of inlets.

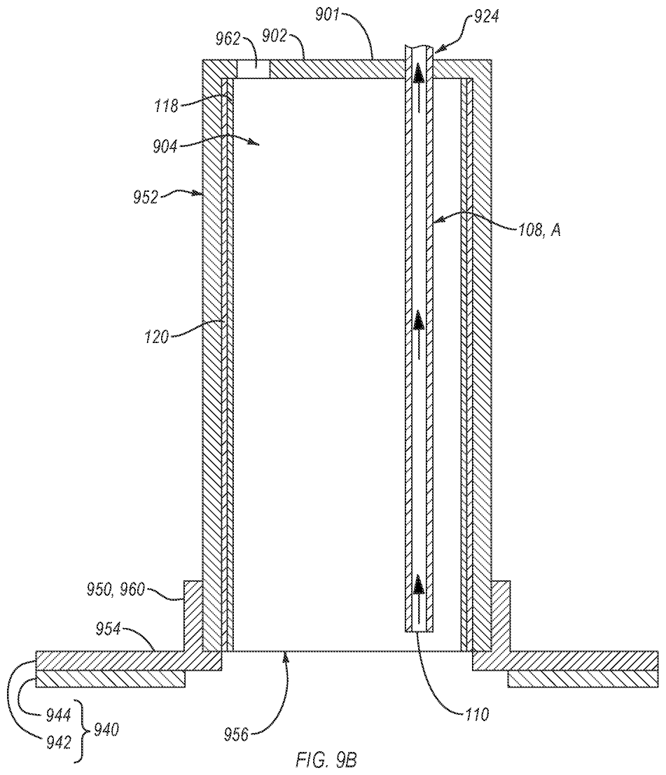

Fluid collection devices having flanges thereon may be configured for use with male users. Devices and methods described herein can be configured to collect urine from a male user, such as having a fluid collection device shaped and sized to receive a male urethra (e.g., penis) therein. FIGS. 9A and 9B are isometric and schematic cross-sectional views of a male fluid collection device 900, according to an embodiment.

Referring to FIGS. 9A and 9B, the fluid collection device 900 includes a receptacle 950 and a cup portion 952. The receptacle 950 is sized, shaped, and made of a material to be coupled to skin that surrounds the male urethra and have the male urethra positioned therethrough. For example, the receptacle 950 can include an annular base 954 that defines an opening 956 in the receptacle 950. The annular base 954 is sized and shaped to be positioned around the male urethra (e.g., positioned around and/or over the penis) and the opening 956 can be configured to have the male urethra positioned therethrough. The annular base 954 can also be sized, shaped, made of a material, or otherwise configured to be coupled (e.g., adhesively attached, such as with a hydrogel adhesive) to the skin around the male urethra (e.g., around the penis) with one or more flanges 940. In examples, the receptacle 950 may include one or more flanges that extend outwardly to contact the wearer of the fluid collection device 900. The annular base 954 may include, define, or be affixed to at least one flange 940 that extends substantially perpendicular to the lip 960.

The flange(s) 940 may be similar or identical to the flange 140 disclosed herein, in one or more aspects. For example, the flange 940 may include the flange body 942 and an adhesive 944 (or other attachment body for attaching the flange body 942 to a subject or clothes of the subject). The flange body 942 may be similar or identical to the flange body 142 disclosed herein, in one or more aspects. For example, the flange body 942 may be formed from a thermoplastic elastomer, polyethylene, polyvinyl chloride, ethylene vinyl acetate, polytetrafluoroethylene, latex, silicon, fabric, woven cloth, etc. The flange body 942 may extend substantially perpendicularly to the lip 960. The adhesive 944 may be similar or identical to the adhesive 144 disclosed herein, in one or more aspects. In some examples, the more than two flanges 940 may extend from the annular base 954. In some examples (not shown), the flange 940 may include a single flange body 942 that extends from and around substantially all of the annular base 954. In such examples, the adhesive 944 may be located at discrete points or around substantially all of the surface of the flange body 942 that is expected to contact the wearer. One or more portions of the receptacle 950 may be formed from the same material as the fluid impermeable barrier 102 such as a fluid impermeable polymer (e.g., silicone, polypropylene, polyethylene, polyethylene terephthalate, polyurethane, a polycarbonate, polyvinyl chloride, latex, silicone, etc.), a metal film, another suitable material, or combinations thereof.

In an example, the annular base 954 can exhibit the general shape of the skin surface that the annular base 954 is selected to be coupled with and/or can be flexible thereby allowing the annular base 954 to conform to any shape of the skin surface. The receptacle 950 also defines a hollowed region that is configured to receive (e.g., seal against) the cup portion 952. For example, the receptacle 950 can include the lip 960 that extends upwardly from the annular base 954. The lip 960 may be tall enough to prevent the cup portion 952 from being accidentally removed from the receptacle 950 (e.g., at least 0.5 cm tall, 1 cm tall, at least 2 cm tall, or at least 5 cm tall). In some examples, the annular base 954 is optional. For example, the receptacle 950 may only include the flange 954. In some examples (not shown), the fluid collection device may have a one piece design, with the cup portion 952 and the receptacle 950 being a single piece. In some examples, the receptacle 950 is optional.

The cup portion 952 includes (e.g., may be formed from) a fluid impermeable barrier 902 that is sized and shaped to fit into the hollowed region of the receptacle 950. The cup portion 952 may be shaped to retain a fluid therein. For example, the fluid impermeable barrier 902 may define the cup portion 952, such as a substantially tubular (e.g., cylindrical) body having an enclosed end as illustrated in FIGS. 9A and 9B. Accordingly, the cup portion 552 may have a generally cupped shape with a chamber 504 therein. The fluid impermeable barrier 902 may be similar or identical to the fluid impermeable barrier 102, in one or more aspects. The fluid impermeable barrier 902 partially defines the chamber 904. The fluid impermeable barrier 902 may also define an opening 956 extending through the fluid impermeable barrier 902 that is configured to have a male urethra positioned therethrough. The fluid impermeable barrier 902 may also include at least one passageway 962 (e.g., vacuum relief hole) that allows the chamber 904 to remain substantially at atmospheric pressure. The at least one passageway 962 may be located at any point on the cup portion 952, such as near or nearer the opening 956. The cup portion 952 also includes at least a portion of the conduit 108 therein, such as at least partially disposed in the chamber 904. For example, the conduit 108 may extend from the cup portion 952 to a region at least proximate to the opening 956. The region proximate to the opening 956 may be disposed near or on the skin around the male urethra (e.g., on the penis). Accordingly, when a patient lays on their back, fluid (e.g., urine) may aggregate near the opening 956 against the skin of the subject. The fluid may be removed from the chamber 904 via the conduit 108. In some examples, the cup portion 952 of the fluid impermeable barrier 902 may be constructed of a material and/or have a thickness that allows the cup portion 952 to collapse when placed under vacuum, such as to remove air around a penis in the fluid collection device 900 during use. In such examples, the conduit 108 may extend only into the chamber 904 at the aperture 924 (e.g., not through to the area adjacent the opening). In such examples, urine may be collected and removed from the fluid collection device 900 at the end nearest the aperture 924. In such examples, the at least one passageway may be located nearest the opening 956.

The fluid collection device 900 may include wicking material therein. The fluid collection device 900 may include the fluid permeable membrane 118. The fluid permeable membrane 118 may be disposed between the fluid impermeable barrier 902 of the cup portion 952 and a penis inserted into the chamber 904. The fluid collection device 900 may include a fluid permeable support 120. The fluid permeable support 120 may be positioned between the cup portion 952 and a penis inserted into the chamber 904, such as between the fluid permeable membrane 118 and the fluid impermeable barrier 902. The sidewalls or the end of the chamber 904 may be covered with one or both the fluid permeable membrane 118 or the fluid permeable support 120.

In some examples, a vacuum source (e.g., vacuum source 16 of FIG. 1) may be remotely located from the cup portion 952. In such examples, the conduit 108 may extend out of and away from the cup portion 952 to the vacuum source (e.g., portable vacuum source). The inlet 110 of the conduit 108 is in fluid communication with the vacuum source, either directly or indirectly. The outlet (not shown) may be in fluid communication with a fluid storage container (not shown) through the conduit 108 in the direction shown by the arrows. The fluid impermeable barrier 902 may include at least one aperture 924 that is sized and shaped to receive and seal against the conduit 108, such as within the chamber 904. Accordingly, the interior region of the chamber 904 may be in fluid communication with the vacuum source 16 via the conduit 108. As the vacuum source applies a vacuum/suction in the direction of the arrows in FIG. 9B, the fluid in the chamber 904 may be removed through the conduit 108. In some examples, the fluid may be pumped via the vacuum source 16 through one or more sections of conduit to the fluid storage container (not shown). In some examples, the vacuum source may be located on or in the cup portion 952 in a manner similar or identical to the vacuum source 330 located within or on the fluid impermeable barrier 102 in FIGS. 4 and 5, in one or more aspects.

In an example, portions of the chamber 904 may be substantially empty due to the varying sizes and rigidity of the male penis. However, in some examples, the outermost regions of the chamber 904 (e.g., periphery of the interior regions of the cup portion 952) can include a porous material (e.g., one or more of the fluid permeable membrane 118 and fluid permeable support 120) positioned (e.g., at the end of the cavity) and configured to blunt a stream of urine from the male urethra, thereby limiting splashing and/or to direct the fluid(s) to a selected region of the chamber 904. Since the chamber 904 is substantially empty (e.g., substantially all of the chamber 904 forms a reservoir), the fluids are likely to pool at a gravimetrically low point of the chamber 904. The gravimetrically low point of the chamber 904 can be at an intersection of the skin of an individual and the fluid collection device 900, a corner formed in the cup portion 952, or another suitable location depending on the orientation of the wearer. The inlet 110 of the conduit 108 can be positioned to be adjacent or proximate to the gravimetrically low point of the chamber 904, such as adjacent to the annular base 954. For example, the inlet 110 may be co-extensive with or offset from the opening 956. In examples, the inlet may be positioned adjacent to the terminal end of the cup portion 952 (e.g., substantially opposite the opening).

During operation, a male using the fluid collection device 900 can discharge fluid(s) (e.g., urine) into the chamber 904. The fluid(s) can pool or otherwise be collected in the chamber 904. At least some of the fluid(s) can enter the interior of the conduit 108 via the inlet 110. The fluid may be drawn out of the fluid collection device 900 via the vacuum/suction provided by the vacuum source. In some examples, during operation, the passageway 962 may substantially maintain the pressure in the chamber 904 at atmospheric pressure even though fluid is introduced into and subsequently removed from the chamber 904.

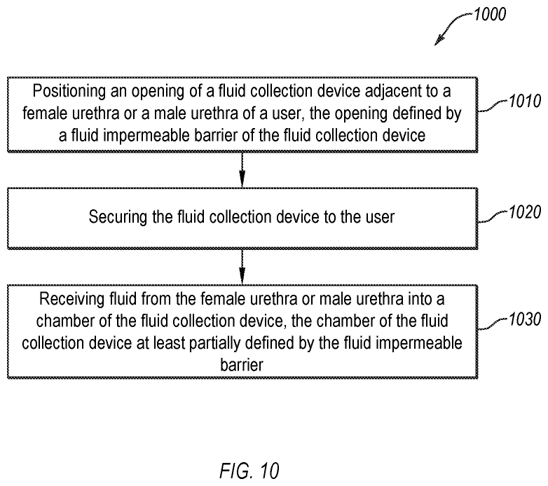

FIG. 10 is a flow diagram of a method 1000 to use any of the fluid collection devices, members, and/or fluid collection systems disclosed herein, according to an embodiment. The method 1000 can include act 1010, which recites “positioning an opening of a fluid collection device adjacent to a female urethra or around a male urethra of a user, the opening defined by a fluid impermeable barrier of the fluid collection device.” Act 1010 may be followed by act 1020, which recites “securing the fluid collection device to the user.” Act 1020 may be followed by act 1030, which recites “receiving fluid from the female urethra or male urethra into a chamber of the fluid collection device, the chamber of the fluid collection device at least partially defined by the fluid impermeable barrier.”

Acts 1010, 1020, 1030 of the method 1000 are for illustrative purposes. For example, the act 1010, 1020, 1030 of the method 1000 can be performed in different orders, split into multiple acts, modified, supplemented, or combined. In an example, one or more of the acts 1010, 1020, 1030 of the method 1000 can be omitted from the method 1000. Any of the acts 1010, 1020, or 1030 can include using any of the fluid collection devices or systems disclosed herein.

Act 1010 recites “positioning an opening of a fluid collection device adjacent to a female urethra or around a male urethra of a user, the opening defined by a fluid impermeable barrier of the fluid collection device.” In some examples, act 1010 can include positioning the opening of a female fluid collection device such that the fluid permeable membrane of the female fluid collection device abuts or is positioned proximate to the female urethra. For example, positioning an opening of a fluid collection device adjacent to a female urethra of a user may include positioning opening of the fluid collection member of the fluid collection device on, adjacent to, between the labia. In examples the female fluid collection device may be similar or identical to the fluid collection device 100, 400, 500, 700, or 800 (FIGS. 2A-8), in one or more aspects.

In some examples, act 1010 can include positioning the opening of a male fluid collection device around a urethra of a male user such that the urethra of the user is positioned within the fluid collection device. For example, positioning an opening of a fluid collection device around a male urethra of a user may include positioning the penis of a user in the cup portion of the male fluid collection device. In examples the male fluid collection device may be similar or identical to the fluid collection device 900 (FIG. 9), in one or more aspects. In such examples, the method 1000 can include positioning a cup portion of the male fluid collection device in a hollowed region of the receptacle such that the male urethra is positioned in the cup portion through the opening in the receptacle of the male fluid collection device. The flanges on the receptacle may be used to secure the fluid collection device to a male user.

Act 1020 recites, “securing the fluid collection device to the user.” Securing the fluid collection device to the user may include affixing one or more flanges of the fluid collection device to the user or clothing of the user. In examples, securing the fluid collection device to the user can include adhering the adhesion member of the at least one flange of the fluid collection device to the user. In examples, securing the fluid collection device to the user can include adhering the adhesion member(s) of the at least one flange (e.g., flanges) of the fluid collection device to one or more of the lower abdomen, inner thigh(s), testicles, pubic region, hip region, or perineum of the user (or on clothing over any of the aforementioned regions). For example, securing the fluid collection device to the user can include adhering the adhesion member(s) of the at least one flange of the fluid collection device to the inner thigh(s) of a female user. Securing the fluid collection device to the user can include adhering the adhesion member of the at least one flange of the fluid collection device to the garment (e.g., the underwear) of the user. For example, adhering the adhesion member of the at least one flange of the fluid collection device to the garment may include adhering one or more flanges on or around the crotch (e.g., gusset) of underwear of the user. Securing the fluid collection device to the user may include securing the fluid collection device to the user to maintain the opening adjacent to, on, or over the urethra of the user, even when the user moves.

Act 1030 recites “receiving fluid from the female urethra or male urethra into a chamber of the fluid collection device, the chamber of the fluid collection device at least partially defined by the fluid impermeable barrier.” In examples, act 1030 can include retaining the fluid within the chamber, such as in a gravimetrically low point therein. In some examples, act 1030 can include wicking the fluid away from the opening using wicking material (e.g., fluid permeable membrane and a fluid permeable support). In some examples, act 1030 can include receiving the fluid into the chamber of the fluid collection device. In either example, act 1030 can include flowing the fluid towards a portion of the chamber that is in fluid communication with an inlet of a conduit in fluid communication with a vacuum source. For instance, act 1030 can include flowing the fluid to a substantially unoccupied portion of the chamber (e.g., a reservoir), to a gravimetrically low point of the chamber, etc. The fluid can include one or more fluids, such as urine, liquid blood, sweat, etc. In some examples, receiving fluid from the female urethra into a chamber of the fluid collection device, the chamber of the fluid collection device at least partially defined by the fluid impermeable bather may include wicking the fluid into the chamber via the fluid permeable membrane and fluid permeable support of the fluid collection device. For example, wicking the fluid into the chamber via the fluid permeable membrane and fluid permeable support may include wicking urine into the reservoir in the fluid collection device.

The method 1000 may include removing at least some of the fluid from the fluid collection device. For example, removing at least some of the fluid from the fluid collection device may include removing the fluid from within the chamber of the fluid collection device. Such removal may include applying suction with a vacuum source effective to suction the fluid from the chamber via a conduit disposed therein (which conduit may be in fluid communication with the vacuum source). In examples, removing fluids from the fluid collection device by applying suction with a vacuum source effective to suction (e.g., vacuum) the fluid from the chamber via a conduit disposed therein include using any of the vacuum sources disclosed herein, such as a portable vacuum source. In an example, applying suction can include activating the vacuum source (e.g., portable suction device) in fluid communication with the inlet of the conduit in the fluid collection device. In examples, activating the vacuum source in fluid communication with the inlet of the conduit in the fluid collection device can include supplying power to the vacuum source by one or more of flipping an on/off switch, pressing a button, opening a valve, connecting the fluid collection device into a vacuum line, plugging a portable vacuum source into a power outlet, putting batteries into the portable vacuum source, etc. In examples, the vacuum source may include a hand operated vacuum pump and applying suction with a vacuum source may include manually operating the hand operated vacuum pump effective to suction the fluid from the chamber via the conduit disposed therein that is in fluid communication with the (portable) vacuum source. In examples, the vacuum source may include a plumbed vacuum line and applying suction with a vacuum source may include manually connecting to the plumbed vacuum line to the fluid collection device (e.g., the conduit) or opening a valve therebetween effective to suction the fluid from the chamber via the conduit disposed therein.

In examples, applying suction with a vacuum source effective to suction the fluid from the chamber via a conduit disposed therein and in fluid communication with the vacuum source can be effective to remove at least some fluid (e.g., urine) from the chamber (e.g., interior region) of the fluid collection device. In examples, applying suction with a vacuum source effective to suction the fluid from the chamber via a conduit disposed therein and in fluid communication with the vacuum source can be effective to transfer at least some of the fluid from the chamber of the fluid collection device to a fluid storage container (e.g., a bottle or bag). In some examples, applying suction with a vacuum source effective to suction the fluid(s) from the chamber may include removing fluid from one or more of a reservoir, fluid permeable support, or fluid permeable membrane of the fluid collection device.

In examples, the vacuum source (e.g., suction device) may disposed on or within the fluid collection device and applying suction with the vacuum source may include activating the vacuum source. In examples, the vacuum source may be spaced from the fluid collection device and applying suction with the vacuum source may include activating the vacuum source, such as a portable vacuum source.

In examples, applying suction with a vacuum source effective to suction the fluid from the chamber via a conduit disposed therein and in fluid communication with the vacuum source may include detecting moisture in the chamber (e.g., via one or more moisture sensors) and responsive thereto, activating the vacuum source to provide suction in the chamber. The control of the vacuum source responsive to the signals indicating that moisture or a level thereof is present in the chamber can be automatic, such as via a controller, or may merely provide an indication that a level of moisture is present that may necessitate removal of fluid from the chamber of the fluid collection device. In the latter case, a user may receive the indication and activate the vacuum source such as a pump.

In an example, the method 1000 can include collecting the fluid that is removed from the fluid collection device, such as into a fluid storage container that is spaced from the fluid collection device and in fluid communication with the conduit. The fluid storage container can include any of the fluid storage containers disclosed herein.

While various aspects and embodiments have been disclosed herein, other aspects and embodiments are contemplated. The various aspects and embodiment disclosed herein are for purposes of illustration and are not intended to be limiting.

We claim:

1. A fluid collection device, comprising:
a fluid collection member, including:
a fluid impermeable barrier at least partially defining a chamber, the fluid impermeable barrier also defining an opening extending therethrough, the opening configured to be positioned adjacent to a female urethra or have a male urethra positioned therethrough;
a porous material disposed at least partially within the chamber; and
at least one flange extending outwardly from the fluid impermeable barrier, the at least one flange including a flange body having an adhesive member thereon.

2. The fluid collection device of claim 1, wherein the at least one flange extends substantially tangentially from the impermeable barrier.

3. The fluid collection device of claim 1, wherein the at least one flange extends substantially perpendicularly from the fluid impermeable barrier.

4. The fluid collection device of claim 1, wherein the fluid impermeable membrane is substantially tubular and the opening through the fluid impermeable barrier exhibits an elongated shape configured to be positioned over the urethra of a wearer.

5. The fluid collection device of claim 1, wherein the porous material includes spun polymer fibers.

6. The fluid collection device of claim 5, wherein the porous material extends across the opening.

7. The fluid collection device of claim 1, further comprising a reservoir defined between the fluid impermeable barrier and the porous material.

8. The fluid collection device of claim 1, wherein the fluid impermeable barrier is elongated and the opening configured to receive a penis therein.

9. The fluid collection device of claim 1, further comprising a conduit disposed within the chamber, the conduit including an inlet positioned within the fluid collection device and an outlet configured to be fluidly coupled to a fluid storage container

10. A fluid collection system, comprising:
a fluid storage container configured to hold a fluid;
a fluid collection device in fluid communication with the fluid storage container, the fluid collection device including:
a fluid collection member, including:
a fluid impermeable barrier at least partially defining a chamber, the fluid impermeable barrier also defining an opening extending therethrough, the opening configured to be positioned adjacent to a female urethra or have a male urethra positioned therethrough;
a porous material disposed at least partially within the chamber; and
a conduit disposed within the porous material, the conduit including an inlet positioned within the fluid collection device and an outlet configured to be in fluid communication with the fluid storage container; and
at least one flange extending outwardly from the fluid impermeable barrier, the at least one flange including a flange body and an adhesive member thereon;
a vacuum source in fluid communication with one or more of the fluid storage container or the fluid collection device, the vacuum source configured to draw fluid from the fluid collection device.

11. The fluid collection system of claim 10, wherein the fluid collection member includes a tubular shape and the opening through the fluid impermeable barrier exhibits an elongated shape configured to be positioned over the urethra of a wearer.

12. The fluid collection system of claim 10, wherein the at least one flange extends substantially perpendicularly from the fluid impermeable barrier.

13. The fluid collection system of claim 10, wherein the at least one flange extends substantially tangentially from the fluid impermeable barrier.

14. The fluid collection system of claim 10, wherein the vacuum source includes a portable vacuum source disposed within the fluid collection device.

15. The fluid collection system of claim 10, wherein:
the fluid collection device is spaced from and positioned upstream from the fluid storage container; and
the vacuum source is positioned downstream from and outside of the fluid collection device.

16. A method to collect fluid, the method comprising:
positioning an opening of a fluid collection device adjacent to a female urethra or a male urethra of a user, the opening defined by a fluid impermeable barrier of the fluid collection device;
securing the fluid collection device to the user with at least one flange extending from the fluid impermeable barrier; and
receiving fluid from the female urethra or male urethra into a chamber of the fluid collection device, the chamber of the fluid collection device at least partially defined by the fluid impermeable barrier and having a porous material therein.

17. The method of claim 16, wherein securing the fluid collection device to the user includes affixing the at least one flange to the user or clothing of the user.

18. The method of claim 16, wherein:
the fluid collection device is configured as a female fluid collection device including:
the fluid impermeable barrier having a tubular shape and the opening has an elongated shape configured to be positioned over the urethra of a wearer;
the at least one flange including a flange body and an adhesive member therein; and
a conduit extending into the chamber; and
securing the fluid collection device to the user includes attaching the at least one flange to skin or clothing of the user.

19. The method of claim 16, wherein:
the fluid collection device is configured as a male fluid collection device having:
the fluid impermeable barrier defining a substantially tubular body configured to receive a penis therein; and
the at least one flange including a flange body and an adhesive member thereon, wherein the flange body is attached to the fluid impermeable barrier; and
a conduit extending into the chamber; and
securing the fluid collection device to the user includes attaching the at least one flange to skin or clothing of the user.

20. The method of claim 16, further comprising removing at least some of the fluid from the fluid collection device with a vacuum source effective to suction the fluid from the chamber via a conduit disposed therein and in fluid communication with the vacuum source.

21. The method of claim 16, wherein the vacuum source is disposed within the fluid collection device and applying suction with the vacuum source includes activating the vacuum source.
